(19) United States
(12) Patent Application Publication
(10) Pub. No.: US 2003/0059375 A1
(43) Pub. Date: Mar. 27, 2003

(54) Method for treating lung cancers

(75) Inventors: Roman Perez-Soler, New York, NY (US); Frank Pilkiewicz, Princeton Junction, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Transave, Inc., Monmouth Junction, NJ

(21) Appl. No.: 10/224,293

(22) Filed: Aug. 20, 2002

Related U.S. Application Data
(60) Provisional application No. 60/313,528, filed on Aug. 20, 2001.
(60) Provisional application No. 60/400,850, filed on Aug. 2, 2002.

(51) Int. Cl. A61L 9/04; A61K 31/337; A61K 33/24; A61K 31/28; A61K 9/127

(52) U.S. Cl. 424/45; 424/450; 424/649; 514/449; 514/492

(57) ABSTRACT

A method of treating bronchoalveolar carcinoma, carcinomatosis with lymphangitic spread or primary and metastatic lung cancers by administering one or more bioactive agents by inhalation of a lipid composition. The one or more bioactive agents preferably comprise cisplatin, carboplatin or a taxane.

This application claims benefit to Provisional Application No. 60/313,528 filed Aug. 20, 2001 and Provisional Application No. 60/400,850 filed Aug. 2, 2002.

The present invention relates to a system for treating bronchoalveolar carcinoma, or carcinomatosis with lymphangitic spread and primary and metastatic lung cancers in general by administering bioactive agents by inhalation. More particularly the present invention relates to the treatment of bronchoalveolar carcinoma, or carcinomatosis with lymphangitic spread, and primary and metastatic lung cancers in general by administering cisplatin, oxaliplatin, carboplatin or a taxane in a lipid composition and by inhalation.

Bronchoalveolar Carcinoma (BAC) or alveolar cell carcinoma is a form of adenocarcinoma, a cell-type of non-small cell carcinoma of the lung which can be found throughout the respiratory tract. BAC represents approximately 10 to 25% of the adenocarcinoma of lung cases or 2-6% of all lung cancers and sometimes has a distinct presentation and biologic behavior. BAC is more common in women and in patients who do not smoke cigarettes than other histologic types of lung cancer

BAC may present as a solitary peripheral nodule, a multifocal lesion, or a rapidly progressive form that appears as a diffuse infiltrate on chest radiograph. The cells secrete mucin and surfactant apoprotein which can lead to bronchorrhea, an excessive discharge of mucus from the air passages of the lungs. Bronchoalveolar cancer may present as a more diffuse lesion than other types of cancer. When it is discovered as a single mass on a patient's x-ray, this type of lung cancer has an excellent prognosis. Five year survival after surgery is in the 75-90 percent range. If, however, it is found in its diffuse form (meaning it has spread beyond a single mass), the prognosis is quite poor.

The management and prognosis are essentially the same as other types of nonsmall cell lung cancer. Surgery is the preferred treatment if the tumor can be resected. Radiation therapy and chemotherapy may be used in non-operable cases. Trials are underway to investigate treatments specific for bronchoalveolar carcinoma.

Carcinomatosis with lymphangitic spread, or Lymphangitis carcinomatosis (LC) refers to the diffuse infiltration and obstruction of pulmonary parenchymal lymphatic channels by tumor. Various neoplasms can cause lymphangitic carcinomatosis, but 80% are adenocarcinomas. The most frequent primary sites are the breast, lungs, colon, and stomach. Other sources include the pancreas, thyroid, cervix, prostate, larynx, and metastatic adenocarcinoma from an unknown primary.

LC occurs as a result of initial hematogenous spread of tumor to the lungs, with subsequent malignant invasion through the vessel wall into the pulmonary interstitium and lymphatics. Tumor then proliferates and spreads easily through these low resistance channels. Less commonly, direct infiltration occurs from contiguous mediastinal or hilar lymphadenopathy or from an adjacent primary bronchogenic carcinoma. Histopathologically, interstitial edema, interstitial fibrosis (secondary to a desmoplastic reaction as a result of tumor extension into adjacent pulmonary parenchyma), and tumor cells all can be seen. Metastatic adenocarcinoma accounts for 80% of cases. Most patients are middle-aged adults

In the U.S. LC represents 7% of all pulmonary metastases. Prevalence in postmortem studies is significantly higher than the incidence of radiologically detectable disease. Microscopic interstitial tumor invasion is seen in 56% of patients with pulmonary metastases. Prognosis for patients with LC is poor. Most patients survive only weeks or months.

The usual presenting complaint is of breathlessness in a patient with known malignancy. Occasionally, patients may have a dry cough or hemoptysis. Symptoms often precede the development of any radiographic abnormality.

There is a continuing need to treat lung diseases such as pre-cancerous or cancerous conditions, damage caused by tobacco smoke and other environmental insults, inflammations and infections.

The lungs can be a portal to the body by means of uptake by cells of the lung such as aveolar macrophages or through the lymphatic system. Administration of drugs through the lung portal for systematic treatment can avoid hepatic first pass inactivation and allow for lower doses with fewer side effects.

In comparison to injection, the administration of a drug by inhalation to treat bronchoalveolar carcinoma, carcinomatosis with lymphangitic spread or primary and metastatic lung cancers where the metastatic spread is primarily within the lungs in general is attractive. Inhalation is a more localized administration of the therapeutic and, therefore, can be more effective. Inhalation can be easier to use. In certain instances the therapeutic can be self-administered leading to better patient compliance and reduced cost. Although inhalation of therapeutics appear to be an attractive alternative to injection for treating lung disease, inhalation administration still has several significant disadvantages: (1) due to the immunology of the lung, drugs that are administered by inhalation quickly clear the lung and, therefore, yield short term therapeutic effects. This rapid clearance can result in the drug having to be administered more frequently and, therefore, adversely affecting patient compliance and increasing the risk of side effects; (2) targeted delivery of the drug by inhalation to the site of cancer is not possible, and the therapeutic is treated like a foreign particle that is quickly cleared from the lung, and eventually it ends up in the reticuloendethial system; (3) inhalation formulations are susceptible to both chemical and enzymatic in-vivo degradation. This degradation is particularly detrimental to peptide and protein formulations; and (4) due to aggregation and lack of stability, formulations of high molecular weight compounds like peptides and proteins are not effectively administered as aerosols, nebulized sprays or as dry powder formulations.

Inhalation is also an attractive option for treatment that involves radiotherapy followed by chemotherapy or chemotherapy followed by radiation therapy. The chemotherapy radiation therapy combination is known to improve the survival rate over radiation treatment alone. Delivery of bioactive agents by inhalation can be a preferred option that allows the treatments to be given closer together, chronologically.

### SUMMARY OF THE INVENTION

The present invention relates to a system for treating bronchoalveolar carcinoma, or carcinomatosis with lymphangitic spread and primary and metastatic lung cancers in general by administering pharmaceuticals by inhalation. The method comprises administering an active compound as part of a lipid composition by inhalation. The method also comprises delivering the lipid composition such that the particles are sized to best deposit in the lungs.

### DETAILED DESCRIPTION OF THE INVENTION

The present invention relates to a system for treating bronchoalveolar carcinoma, or carcinomatosis with lymphangitic spread, primary and metastatic lung cancers in general and radiotherapy followed by chemotherapy (particularly for lungs and lung cancers both primary and metastatic) by administering pharmaceuticals by inhalation. The pharmaceuticals are administered as part of a lipid or liposome composition.

The compositions can include liposomes, lipid complexes, lipid clathrates and proliposomes, i.e., compositions which can form liposomes in vitro or in vivo when contacted with water. Compositions are preferably adopted for use by inhalation, and more preferably for use in an inhalation delivery device for the composition's administration. The inhalation system can be used for the treatment of lung cancers in both man and animal.

Bioactive agents can include radiocontrast agents, such as the iodinated radiocontrast agents, for example, iotrolan, NMR contrast agents, radioisotopes, radiolabels and dyes. The above-listed group of bioactive agents, among other agents, including their pharmaceutically acceptable salts, are contemplated for use in the present invention. Determination of compatibilities of the above listed agents with, and the amounts to be utilized in, compositions of the present invention are within the purview of the ordinarily skilled artisan to determine given the teachings of this invention.

The lipids used in the compositions of the present invention can be synthetic, semi-synthetic or naturally-occurring lipids, including phospholipids, tocopherols, sterols, fatty acids, glycoproteins such as albumin, negatively-charged lipids and cationic lipids. In terms of phosholipids, they could include such lipids as egg phosphatidyl choline (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylglycerol (DMPG) dipalmitoylphosphatidcholine (DPPQ and dipalmitoylphosphatidylglycerol (DPPG) distearoylphosphatidylcholine (DSPQ and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolarnine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (PIs) and the phosphatidyl serines (PSs). Examples include DMPG, DPPG, DSPG, DMPA, DPPA, DSPA, DMPI, DPPI, DSPI, DMPS, DPPS and DSPS.

Phosphatidylcholines, such as DPPC, aid in the uptake by the cells in the lung (e.g., the alveolar macrophages) and helps to sustain release of the bioactive agent in the lung. The negatively charged lipids such as the PGs, PAs, PSs and PIs, in addition to reducing particle aggregation, are believed to play a role in the sustained release characteristics of the inhalation formulation as well as in the transport of the formulation across the lung (transcytosis) for systemic uptake. The sterol compounds are believed to affect the release characteristics of the formulation.

In general, PE's such as DOPE, DMPE, DPPE, DSPE and MOPE can be employed in the lipid mixtures of the present invention.

For lipid mixtures, particularly for use with biologically active compounds of high molecular weight ( e.g., peptides, proteins, DNA, RNA, genes), a glycoprotein such as albumin or transferring, referred to as an “albumin compound” can be present. The albumin compounds can be present at a mole ratio of 0.1 to 10 with respect to the other lipids. For example, a lipid mixture can be DPPC: DMPG: albumin in a 8:1:2 mole ratio. The albumin can come from either natural, animal (e.g., human or bovine serum albumin) or synthetic sources.

Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the 60 mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980 Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

In a liposome-drug delivery system, a bioactive agent such as a drug is entrapped in the liposome and then administered to the patient to be treated. For example, see Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Alternatively, if the bioactive agent is lipophilic, it may associate with the lipid bilayer. In the present invention, the term “entrapment” shall be taken to include both the drug in the aqueous volume of the liposome as well as drug associated with the lipid bilayer.

A liposome's size is typically referred to in terms of its diameter, and can be measured by a number of techniques well known to ordinarily skilled artisans, such as quasi-electric light scattering. In the present invention the liposomes generally have a diameter of greater than about I micron and up to 5 microns, preferably greater than 1 to 3 microns.

Liposomes sizing can be accomplished by a number of methods, such as extrusion, sonication and homogenization techniques which are well known, and readily practiced, by ordinarily skilled artisans. Extrusion involves passing liposomes, under pressure, one or more times through filters having defined pore sizes. The filters are generally made of polycarbonate, but the filters may be made of any durable material which does not interact with the liposomes and which is sufficiently strong to allow extrusion under sufficient pressure. Preferred filters include “straight through” filters because they generally can withstand the higher pressure of the preferred extrusion processes of the present invention. “Tortuous path” filters may also be used. Extrusion can also use asymmetric filters, such as AnotecO filters (see Loughrey et al., U.S. Pat. No. 5,059,421), which involves extruding liposomes through a branched-pore type aluminum oxide porous filter.

Liposomes can also be size reduced by sonication, which employs sonic energy 5 to disrupt or shear liposomes, which will spontaneously reform into smaller liposomes. Sonication is conducted by immersing a glass tube containing the liposome suspension into the sonic epicenter produced in a bath-type sonicator. Alternatively, a probe type sonicator may be used in which the sonic energy is generated by vibration of a titanium probe in direct contact with the liposome suspension. Homogenization and milling apparatii, such as the Gifford Wood homogenizer, Polytron™ or Micro fluidizer™, can also be used to break down larger liposomes into smaller liposomes.

The resulting liposomes can be separated into homogeneous populations using methods well known in the art; such as tangential flow filtration (see WO 89/00846). In this procedure, a heterogeneously sized population of liposomes is passed through tangential flow filters, thereby resulting in a liposome population with an upper and/or lower size limit. When two filters of differing sizes, that is, having different pore diameters, are employed, liposomes smaller than the first pore diameter pass through the filter. This filtrate can the be subject to tangential flow filtration through a second filter, having a smaller pore size than the first filter. The retentate of this filter is a liposome population having upper and lower size limits defined by the pore sizes of the first and second filters, respectively.

Mayer et al. found that the problems associated with efficient liposomal entrapment of lipophilic ionizable bioactive agents such as antineoplastic agents, for example, anthracyclines or vinca alkaloids, can be alleviated by employing transmembrane ion gradients (see PCT application 86/01102, published Feb. 27, 1986). Aside from inducing greater uptake, such transmembrane gradients also act to increase drug retention in the liposomes.

Liposomes themselves have been reported to have no significant toxicities in previous human clinical trials where they have been given intravenously. Richardson et al., (1979), Br. J. Cancer 40:35; Ryman et al., (1983) in “Targeting of Drugs” G. Gregoriadis, et al., eds. pp 235-248, Plenum, N.Y.; Gregoriadis G., (1981), Lancet 2:241, and Lopez-Berestein et al., (1985). Liposomes are reported to concentrate predominately in the reticuloendothelial organs lined by sinosoidal capillaries, i.e., liver, spleen, and bone marrow, and phagocytosed by the phagocytic cells present in these organs.

The therapeutic properties of many agents can be dramatically improved by the administration in a liposomally encapsulated form (See, for example, Shek and Barber (1986)). Toxicity can be reduced, in comparison to the free form of the drug, meaning that a higher dose of the liposomally encapsulated drug can safely be administered (see, for example, Lopez-Berestein, et al. (1985) J. Infect. Dis., 151:704; and Rahman, et al. (1980) Cancer Res., 40:1532). Benefits obtained from liposomal encapsulation likely result from the altered pharmacokinetics and biodistribution of the entrapped drug.

A number of methods are presently available for “charging” liposomes with bioactive agents (see, for example, Rahman et al., U.S. Pat. No. 3,993,754; Sears, U.S. Pat. No. 4,145,410; Papahadjopoulos, et al., U.S. Pat. No. 4,235,871; Lenk et al., U.S. Pat. No. 4,522,803; and Fountain et al., U.S. Pat. No. 4,588,578). Ionizable bioactive agents have been shown to accumulate in liposomes in response to an imposed proton or ionic gradient (see, Bally et al., U.S. Pat. No. 5,077,056; Mayer, et al. (1986); Mayer, et al. (1988); and Bally, et al. (1988)). Liposomal encapsulation could potentially provide numerous beneficial effects for a wide variety of bioactive agents and a high bioactive agent to lipid ratio should prove important in realizing the potential of liposomally encapsulated agents.

For the references disclosed herein, their disclosures are incorporated herein by reference.

A “lipid complex” is an association between a bioactive agent and one or more lipids. The association can be by covalent or ionic bonding or by noncovalent interactions. Examples of such complexes include lipid complexes of amphotericin B and cardiolipin complexed with doxorubicin.

A “lipid clathrate” is a three-dimensional, cage-like structure employing one or more lipids wherein the structure entraps a bioactive agent.

“Proliposomes” are formulations that can become liposomes upon coming in 5 contact with an aqueous liquid. Agitation or other mixing can be necessary.

The inhalation delivery device of the inhalation system can be a nebulizer, a metered dose inhaler (MDI) or a dry powder inhaler (DPI). The device can contain and be used to deliver a single dose of the lipid mixed—bioactive agent compositions or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer is employed to ensure the sizing of particles is optimal for positioning of the particle within, for example, the lungs.

A metered dose inhalator (MDI) can be employed as the inhalation delivery device of the inhalation system. This device is pressurized (pMDI) and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid based formulation. To insure accuracy of dosing, the delivery of the formulation may be programmed via a microprocessor to occur at a certain point in the inhalation cycle. The MDI may be portable and hand held.

A dry powder inhalator (DPI) can be used as the inhalation delivery device of the inhalation system. This device's basic design consists of a metering system, a powdered composition and a method to disperse the composition. Forces like rotation and vibration can be used to disperse the composition. The metering and dispersion systems may be mechanically or electrically driven and may be microprocessor programmable. The device may be portable and hand held. The inhalator may be multi or single dose in design and use such options as hard gelatin capsules, and blister packages for accurate unit doses. The composition can be dispersed from the device by passive inhalation; i.e., the patient's own inspiratory effort, or an active dispersion system may be employed. The dry powder of the composition can be sized via processes such as jet milling, spray dying and supercritical fluid manufacture. Acceptable excipients such as the sugars mannitol and maltose may be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The sugar by its hydroxyl groups may help the vesicles maintain their tertiary hydrated state and help minimize particle aggregation.

These three general types of inhalation delivery devices can also be used to deliver the vaccine compositions of the present invention.

Some specific examples of bioactive agents that can be present in the compositions of the inhalation system and the uses of the system in the treatment of bronchoalveolar carcinoma, or carcinomatosis with lymphangitic spread, primary and metastatic lung cancers in general and radiotherapy followed by chemotherapy (particularly for lungs and lung cancers both primary and metastatic) include: anticancer agents listed above for lung cancer in particular active platinum compounds such as cisplatin, oxaliplatin, carboplatin, iproplatin, tetraplatin, transplatin, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)) and JM335 (trans-amminedichloro(cyclohexylamine)dihydroxoplatinum(IV)) and taxanes such as paclitaxel and docetaxel.

The pharmaceutical formulation of the inhalation system may contain more than one pharmaceutical (e.g., two drugs for a synergistic effect).

In addition to the above discussed lipids and albumin and drug(s), the composition of the pharmaceutical formulation of the inhalation system may contain excipients (including solvents, salts and buffers), preservatives and surfactants that are acceptable for administration by inhalation to humans or animals.

The particle size of the pharmaceutical formulations developed for use in the inhalation system can vary and be between 0.5 and 10 microns, with a range of 1 to 5 microns being best suited for inhalation and a range of approximately 1 to 2 microns being best suited to deposition in the lungs. Some specific examples of bioactive agents that can be present in the compositions of the inhalation system and the uses of the system in the treatment of bronchoalveolar carcinoma, or carcinomatosis with lymphangitic spread, primary and metastatic lung cancers in general formulation of the inhalation system may be present as a powder, a liquid, or as a suspension.

The term “treatment” or “treating” means administering a composition to an animal such as a mammal or human for preventing, ameliorating, treating or improving a medical condition.

In general, the doses of a bioactive agent will be chosen by a physician based on the age, physical condition, weight and other factors known in the medical arts. Generally for bioactive agents, the dosages will be within the same employing the present invention as for the free drug.

### References for Inhalation Device

1. Hickey A. J. and Dunbar C. A., “A New Millennium for Inhaler Technology”, Pharmaceutical Technology, pgs. 119-125, June (1997).

2. P. Lloyd et. al., “A New Unit Dose, Breath-Actuated Aerosol Drug Delivery System,” in Respiratory Drug Delivery V, P. R. Byron, R. N. Dalby and S. J. Farr, Eds., Interpharm. Press, Buffalo Grove, pgs. 364-366 (1996).

3. J. H. Bell, P. S. Hartley and J. S. G. Cox, “Dry Powder Aerosols I: A New Inhalation Device,” J. Pharm. Sci., 60 (10), pgs. 1539-1564 (1971).

4. J. W. Tom and P. G. Debendetti, “Particle Formation with Supercritical Liquids-A Review,” J. Aerosol Sci., 22, pgs. 555-584 (1991).

5. M. P. Billings, R. N. Boyes, L. M. Clisby, A. E. Harper, P. Braithwaite and S. Williams, “Design, Development and Performance of a Novel Multidose Dry Powder Inhaler,” Pharmaceutical Technology, pgs. 70-82, October (1999).

6. Ogden J., Rogerson C. and Smith I., “Issues in Pulmonary Delivery”, Scripps Magazine, pgs. 56-60, June (1997).

### Other References

1. Possmayer F. I., Metcalfe I. L. and Ehorning G., The Pulmonary Surfactant-Contol of Fluidity at the Air-Liquid Membrane, In:: Membrane Fluidity, Kates M. and Katis A. (eds.), pgs. 57-67, Clifton N.J., Humana Press (1980).

2. Ligand Targeting of Liposomes, Leserman L. and Machy P., In:: Liposomes from Biophysics to Therapeutics, pgs 157-194, Ostro M. (ed.), New York, N.Y., Marcel Dekker (1987).

3. Gonzalez-Rothi R. J., Casace J., Straub L. and Schreier H., Liposomes and Pulmonary Alveolar Macrophages: Functional and Morphologic Interactions, Exp. Lung Res., 17, pgs. 687-705 (1991).

4. Geiger K., Gallagher M. L. and Hedley-White J., Cellular Distribution and Clearance of Aerosolized Dipalmitoyl Choline, J. Appl. Physiol., 39, pgs. 759-766(1975).

5. Bates S. R., Ibach P. B. and Fisher A. B., Phospholipids Co-Isolated with Rat Protein C Account for the Apparent Protein Enhanced Uptake of Liposomes into Lung Granular Pneumocytes, Exp. Lung Res., 15, pgs. 695-708 (1989).

6. A. G. Goodman, and L. Goodman “The Pharmacological Basis of Therapeutics, Eighth Edition” A. G. Goodman, T. W. Rall, A. S. Nies and P. Taylor, Editors, Permagon Press, New York, N.Y. (1990).

What is claimed:

1. A method of treating bronchoalveolar carcinoma, carcinomatosis with lymphangitic spread or primary and metastatic lung cancers, the method comprising administering one or more bioactive agents by inhalation of a lipid composition.

2. The method of claim 1, wherein the one or more bioactive agents comprise cisplatin, oxaliplatin, carboplatin or a taxane.

3. The method of claim 1, wherein the one or more bioactive agents comprise cisplatin.

4. The method of claim 1, wherein the one or more bioactive agents comprise carboplatin.

5. The method of claim 1, wherein the one or more bioactive agents comprise oxaliplatin.

6. The method of claim 1, wherein the one or more bioactive agents comprise a taxane.

7. The method of claim 6, wherein the taxane is paclitaxel.

8. The method of claim 6, wherein the taxane is docetaxel.

9. The method of claim 1, wherein the lipid composition is a liposome.

10. The method of claim 1, wherein the step of administering the lipid composition comprises nebulizing the lipid composition and delivering resulting particles to the lungs of a patient.

11. The method of claim 10, wherein the particles are between 0.5 and 10 microns in diameter.

12. The method of claim 10 wherein the particles are between 1 and 2 microns in diameter.
